(19) United States
(12) Patent Application Publication
(10) Pub. No.: US 2005/0053200 A1
(43) Pub. Date: Mar. 10, 2005

(54) Intra-operative CT scanner

(76) Inventors: Predrag Sukovic, Birmingham, MI (US); Neal Clinthorne, Ann Arbor, MI (US); Nathaniel Bair, Ann Arbor, MI (US)

Correspondence Address: CARLSON, GASKEY & OLDS, P.C., 400 WEST MAPLE ROAD, SUITE 350, BIRMINGHAM, MI, 48009, US

(21) Appl. No.: 10/914,610

(22) Filed: Aug. 9, 2004

Related U.S. Application Data
(60) Provisional application No. 60/493,376, filed on Aug. 7, 2003.

(51) Int. Cl. H05G 1/00; A61B 5/05

(52) U.S. Cl. 378/210

(57) ABSTRACT

A surgical system includes a surgical instrument and a tracking system. The tracking system determines the position and orientation of the surgical instrument relative to the patient and relative to a 3D image of the patient. The relative locations of the surgical instrument and 3D image are shown on a display. The tracking system also determines the position and orientation of a CT scanner that takes x-ray update images of a selected area of interest of the patient. A computer stores data at least partially representing an area of a patient. The computer generates a 3D image or model of the area of interest based upon the update images of the area of interest and based upon the data. The area of interest can be selected on the computer display, automatically based upon the tracked positions of the surgical instrument, or through the use of an interest indicator instrument. The surgeon places the tip of the interest indicator instrument near the physical area of interest and then activates a switch or button on the interest indicator instrument. The area of interest is then defined as an area surrounding the tip of the interest indicator instrument at the time the switch was activated.

This application claims priority to U.S. Provisional Ser. No. 60/493,376, filed Aug. 7, 2003.

### BACKGROUND OF THE INVENTION

The present invention relates to a system utilized for image guided surgery and more particularly to a system which updates pre-operative data with data collected from an intra-operative scan.

Image guided surgery is becoming more common, especially in the areas of intracranial surgery. Systems are utilized to take data gathered from pre-operative scans by MRI, CT scanners, ultrasounds, or the like. The data is used to generate two and three-dimensional images to guide a surgeon during an operation. Often this includes some method for tracking an instrument location with respect to the image displayed by the system. Generally, the image is registered relative to locators attached to the patient. Then, the position and orientation of the surgical instruments is registered and tracked relative to the image and the patient so that the location and orientation of the instruments relative to the patient and the image is continuously displayed.

The problem with using the pre-operative image is that the object selected may have shifted between the time the pre-operative image was taken and the time of surgery. This is especially true once surgery has begun and the shape of the intracranial cavity changes as the surgeon gains access. Changes in the pre-operative image and the actual surgical subject introduce variations into the surgical process. In matters like intracranial surgery the tolerance for variations is low, thus even small changes between the image and actual subject may cause problems and make the surgery less effective. To solve this problem additional images may be taken during surgery to update the previously received information. However, selecting the area to be scanned, setting up the intraoperative scanner, and performing the scan require movement of bulky equipment and surgery must be stopped to set up the equipment properly and perform the scan. In addition it is difficult to move the equipment to the desired area to be scanned thus increasing the time and effort required to update the image properly.

Accordingly, it is desirable to provide a system that aids the surgeon in selecting the area of interest to be scanned and provides an easier method to set up and perform the scan.

### SUMMARY OF THE INVENTION

A surgical system includes a surgical instrument and a tracking system. The tracking system determines the position and orientation of the surgical instrument relative to the patient and relative to a 3D image of the patient. The relative locations of the surgical instrument and 3D image are shown on a display. The tracking system also determines the position and orientation of a CT scanner that takes x-ray update images of a selected area of interest of the patient. A computer stores preoperative data at least partially representing an area of a patient. The computer generates a 3D image or model of the area of interest based upon the update images of the area of interest and based upon the preoperative data.

The area of interest can be selected via a computer graphical user interface, automatically based upon the tracked positions of the surgical instrument, or through the use of an interest indicator instrument. The surgeon places the tip of the interest indicator instrument near the physical area of interest and then activates a switch or button on the interest indicator instrument. The area of interest is then defined as an area surrounding the tip of the interest indicator instrument at the time the switch was activated.

### BRIEF DESCRIPTION OF THE DRAWINGS

Other advantages of the present invention can be understood by reference to the following detailed description when considered in connection with the accompanying drawings wherein:

FIG. 1 illustrates a first embodiment of the intraoperative scanning and navigation system of the present invention.

FIG. 2 illustrates a second embodiment of the intraoperative scanning and navigation system of the present invention.

FIG. 3 is a more detailed view of the CT scanner of FIGS. 1 and 2.

### DETAILED DESCRIPTION OF THE PREFERRED EMBODIMENTS

A first embodiment of an intraoperative scanning and navigation system 10 according to the present invention includes an intra-operative CT scanner 11, a tracking system 12 and surgical instruments 14 (one shown) having fiducials or locators 16.

Prior to surgery, the tracking system 12 tracks the position and orientation of the patient 20 during a preoperative scan based upon the locators 16, so that the position and orientation of the patient 20 relative to the preoperative scan is known. Alternatively, the CT scanner 11 or MRI may register the positions of the locators 16 on the patient 20 relative to the pre-operative scan (CT or MRI) based upon the appearance of the locators 16 in the pre-operative images. As yet another alternative, for some types of surgery, it is possible that some generic data (i.e. data that is not specifically from the present patient) describing the area surrounding the area of interest may be sufficient. The preoperative data may be a complete 3D image or model of the area surrounding an area of interest of the patient, or the preoperative data may be a partial 3D image of the area.

However this preoperative data is acquired, once in the operating room, the patient 20 is registered by the tracking system 12 relative to the pre-operative 3D image. Several types of suitable tracking systems 12 are known. The tracking system 12 may include sensors 25, which may be CCDs that optically detect the locators 16 or RF receivers that receive wireless signals from the locators 16. Other types of tracking systems 12 could also be utilized. The present invention is independent of the specific type of tracking system 12 used.

The tracking system 12 determines the position and orientation of the surgical instruments 14 based upon the locators 16. The tracking system 12 also determines the position and orientation of the patient 20 with the locators 16, such that the position and orientation of the preoperative data (whether or not a complete 3D image), a 3D model of the area (as updated), the surgical instruments 14, and the patient 20 are all known relative to one another. The relative locations of the surgical instruments 14 and the 3D model of the area are displayed on a display 22 of a computer 24.

The CT scanner 11 includes an x-ray source 26 and x-ray detector 27 spaced apart by a gantry 28 which is mounted to rotate at least partially about the patient 20 while taking a plurality of x-ray images of the patient 20 at a plurality of rotational positions. Suitable CT scanners 11 are known, but would preferably utilize a cone-beam x-ray source 26 and a flat-panel detector 27 having a converter for converting x-rays to visible light and an array of photodetectors behind the converter. Any suitable source 26 and detector 27 could be utilized, as the invention is independent of the specific technology used for the CT scanner 11.

The CT scanner 11 takes intraoperative images of an area of interest of the patient 20. The CT scanner 11 may scan the locators 16 too, so that the position and orientation of the images from the CT scanner 11 relative to the patient 20, surgical instruments 14 and preoperative data can be determined. The CT scanner 11 may also include a plurality of locators 16 on the source 26, detector 27 and/or gantry 28 so that the position and orientation of the CT scanner 11 may be registered relative to the preoperative data, the current 3D image, the patient 20 and the surgical instruments 14 in that manner.

For intraoperative scans, the CT scanner 11 only scans an area of interest, which may be the area where the surgeon is working and thus the only area where updates are necessary. The pre-operative data (CT, MRI or generic) is used by the computer 24 in conjunction with the new information from the intraoperative CT scans to convert the intraoperative CT scans into an updated intraoperative 3D image. For example, the preoperative data regarding the areas surrounding the area of interest is needed when updating the image of the area of interest because the data surrounding the area of interest must be deducted from the intraoperative CT scans in order to build the current image of the area of interest. Therefore, the system does not require full intraoperative CT scans to form the image of the selected area of interest. The smaller area of interest scans reduce the dosage of x-rays experienced by the patient 20 compared to the dosage of performing another full scan.

The surgeon can select and define the area of interest relative to the preoperative or intraoperative image on the display 22, using a computer mouse 30 or other input device. The surgeon may confirm that the scanned image will properly be focused on an area of interest to the surgeon. Alternatively, because the locations of the surgical instruments 14 are tracked, the computer 24 can determine the area of interest based upon the previous locations of the surgical instruments 14 while the surgery was performed. In other words, the area of interest may be defined by the computer 24 to be the area within some predetermined distance of the positions of the surgical instruments 14 within the patient 20 (as determined by the tracking system 12 and the preoperative or previously updated 3D images) because those are the areas most likely to have changed and to need an updated image. After continuously tracking the positions of the surgical instruments 14 within the patient 20, the surgeon can tell the computer 24 via mouse 30 (or other input device) to perform the update scan. Alternatively, the computer 24 can display the proposed area of interest on the display 22, which the surgeon can review, modify and approve before performing the update scan.

The surgeon also has the option of using an interest indicator instrument 36 having a tip 38 and locators 16 to indicate the area of interest to be updated by the CT scanner 11. With this option, the surgeon places the tip 38 of the interest indicator instrument 36 in the area where the surgeon wants the 3D image updated. The tracking system 12 monitors the location and orientation of the interest indicator instrument 36. When the surgeon activates a button 40 on the interest indicator instrument 36, a signal is sent to the tracking system 12, telling the tracking system 12 to record the current location of the tip 38 of the interest indicator instrument 36 as the area of interest. When the surgeon requests the update to start (via computer 24), the computer 24 controls the CT scanner 11 to move to the necessary location and to collimate the x-ray source 26 to the area of interest to obtain the updated information. The CT scanner 11 then performs an update scan of the area of interest using a small field of view, which limits the dosage experienced by the patient 20. The 3D image on computer 24 is then updated (or re-created) based upon the update scan, the pre-operative 3D image (or the pre-operative individual 2D images) and possibly based upon previous updates to this or other areas of interest as well.

As stated above, the surgical instruments 14 are also registered relative to the imaging system. By registering the instrument 14 the system can then locate the instrument 14 and the relative position and orientation of the instrument 14 to the area of interest can be displayed on the display 22. In the manner, the surgeon can perform navigation-guided surgery in a known manner.

Since the pre-operative data is used only for background information and calculations required in creating a new image, the pre-operative data has lesser importance in the new image than does the intra-operative data. As a result, the pre-operative scan may use a lower dosage and/or lower resolution than would otherwise be used. The result is a safer pre-operative scan for the patient 20 and a cost savings in obtaining the pre-operative scan.

The preoperative and/or updated intraoperative 3D image may also be used in conjunction with an intra-operative fluoroscope comprising a fluoroscopy source 44 and detector 45. The position and orientation of the fluoroscope 44, 45 is repeatedly registered relative to the pre-operative and intraoperative images in the manner described above. The fluoroscope 44, 45 provides an image that is constantly being updated. The intra-operative fluoroscopy image created from the scan may focus on areas of interest to provide the surgeon with data sufficient for guiding the surgery. The constant scanning provides the surgeon with an accurate image of the area of interest, which reflects changes in the patient conditions. The surgeon may rotate and position the 3D image from the CT scanner 11 on the computer display 22 and select or change a desired area of interest to be scanned by the fluoroscope 44, 45. The surgeon can select and define the area of interest on the computer display 22 using a computer input device such as a mouse, or by using the interest indicator instrument 36 in the manner described above. The fluoroscope 44, 45 is then moved by motors controlled by the computer 24 to the correct position and orientation to scan the area of interest. Alternatively, the surgeon can manually move the fluoroscope 44, 45 while watching the fluoroscopy image superimposed on the 3D CT image in the current position and orientation on the display 22 until the fluoroscopy source 44 is scanning the desired area of interest. Alternatively, the CT scanner 11 can be used to perform the fluoroscopy as well, simply by using the x-ray source 26 as the fluoroscopy source and the detector 27 to continuously receive the x-rays from the x-ray source 26 and generate a continuously updated 2D image on the display 22 in the manner described above.

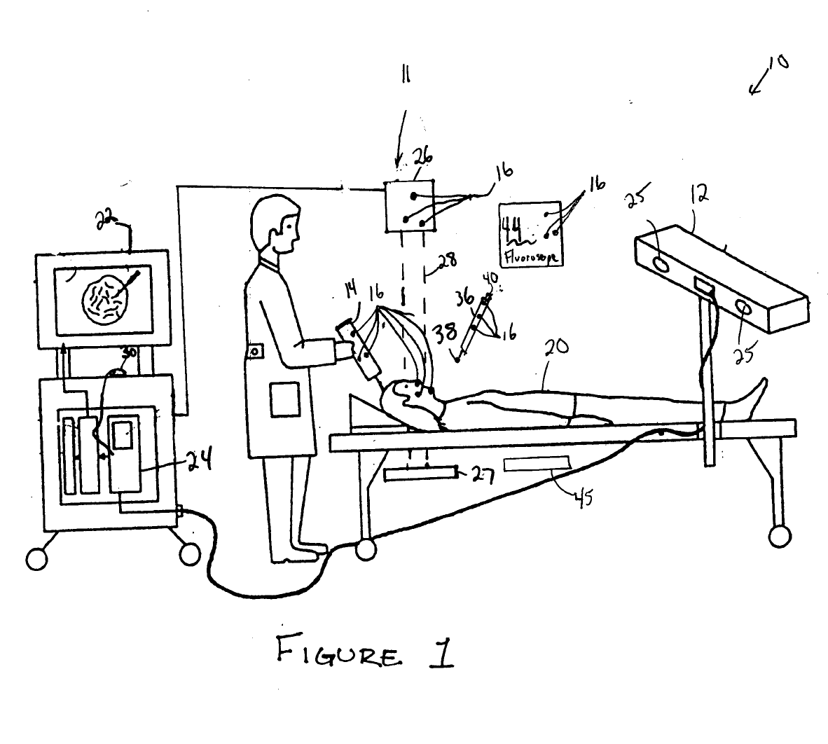

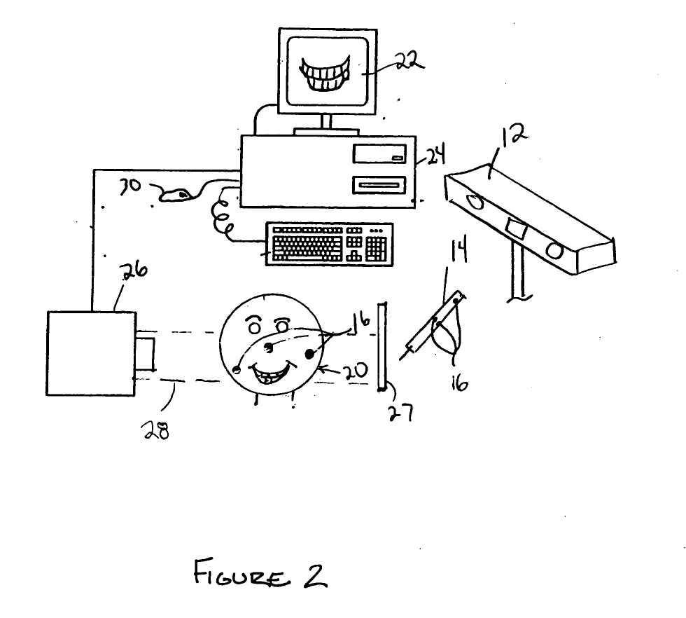

In another embodiment of the present invention, a pre-operative CT scan is used to assist during dental surgery, specifically image guided dental implantology. This is illustrated in FIG. 2. The CT scanner 11 takes a preoperative scan of the patient 20 with locators 16 attached to the patient. The surgical instruments 14 (one shown) to be used during the surgery are registered with the tracking system 12. After the instrument 14 is registered with the tracking system 12 the location of the surgical instrument 14 is tracked by the tracking system 12. The computer 24 tracks the location of the instrument 14 relative to the preoperative scan and relative to the patient 20. The display 22 displays the current location of the instrument 14 on the preoperative image. The dental surgeon then has the ability to view the instrument 14 relative to the image. The preoperative image can be updated in the manner described above, including using the graphical user interface, automatically based upon the locations of the instrument 14 or via the interest indicator instrument 36 (FIG. 1).

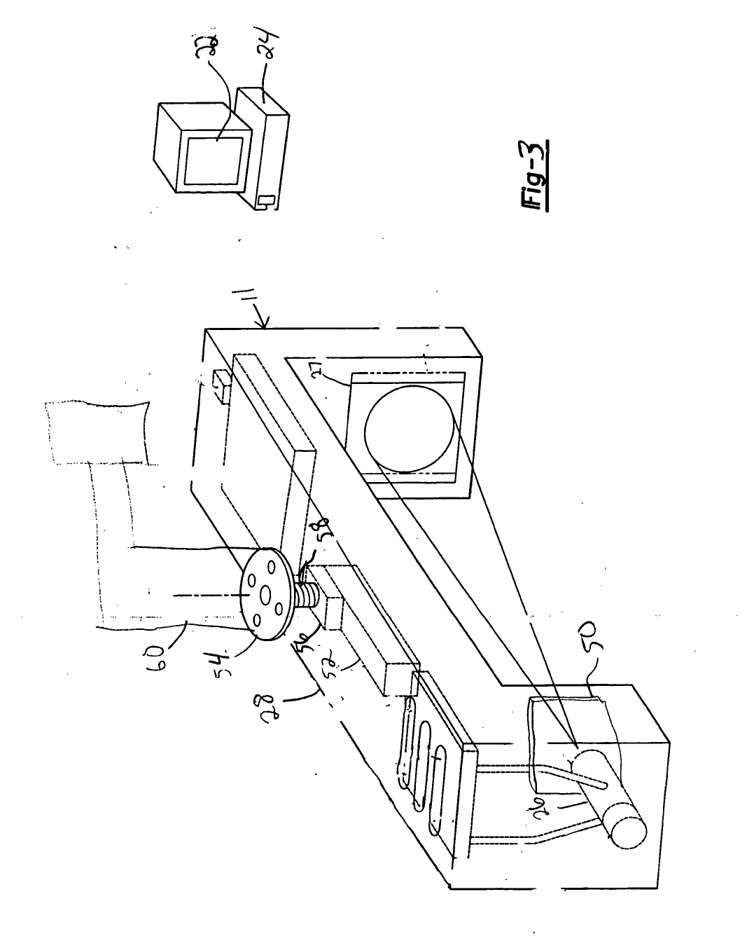

FIG. 3 illustrates one possible configuration of a CT scanner 11 that could be used as the CT scanner 11 of FIGS. 1 and 2 (horizontally in FIG. 1 and vertically in FIG. 2). The x-ray source 26 and detector 27 are mounted at opposite ends of the gantry 28. A collimator 50 is mounted in the gantry 28 in front of the x-ray source 26. A motor 52 is mounted in the gantry 28 for rotating the gantry 28 relative to a mounting plate 54. The motor 52 may directly drive the mounting plate 54, or a gear box 56 may be provided between the motor 52 and mounting plate 54. As an additional option, a ball screw 58 may be provided between the motor 52 and mounting plate 54 for providing some translation of the gantry 28 along the axis of rotation of the motor 52. For example, the ball screw 58 would provide approximately 1 inch of translation in one complete rotation of the gantry 28. The mounting plate 54 may be mounted to a motor-controlled arm 60 for relocating the gantry 28 to the patient 20 and to the area of interest. The motor 52 and arm 60 are controlled by the computer 24.

In accordance with the provisions of the patent statutes and jurisprudence, exemplary configurations described above are considered to represent a preferred embodiment of the invention. However, it should be noted that the invention can be practiced otherwise than as specifically illustrated and described without departing from its spirit or scope.

1. A method for updating an image of a patient including the steps of:
a) storing first image data representing a first area of a patient;
b) determining an area of interest;
c) collecting second image data from a scan of a second area of the patient, the second area including the area of interest, the second area of the patient smaller than the first area of the patient; and
d) creating a 3D image of at least the area of interest based upon the second image data and at least a portion of the first image data.

2. The method of claim 1 wherein the area of interest is determined in said step b) based upon a location of an instrument.

3. The method of claim 2 wherein the instrument is a surgical instrument.

4. The method of claim 2 wherein the location of the instrument is tracked through a plurality of locations and wherein the area of interest includes the plurality of locations.

5. The method of claim 1 wherein said step b) includes the step of determining a location of an instrument and determining the area of interest based upon the location of the instrument.

6. The method of claim 5 wherein said step b) further includes the step of placing the instrument at the location and selectively designating the location as the area of interest.

7. The method of claim 1 wherein said step a) includes the step of taking a first plurality of x-ray images at a first plurality of rotational positions about the patient.

8. The method of claim 7 wherein said step c) includes the step of taking a second plurality of x-ray images at a second plurality of rotational positions about the patient.

9. The method of claim 8 wherein said step a) includes the step of constructing a 3D image of the first area of the patient.

10. The method of claim 9 wherein the area of interest is determined relative to the 3D image in said step b).

11. The method of claim 10 wherein the area of interest is determined in said step b) based upon a location of an instrument.

12. The method of claim 11 wherein the instrument is a surgical instrument.

13. The method of claim 11 wherein the location of the instrument is tracked through a plurality of locations and wherein the area of interest includes the plurality of locations.

14. The method of claim 10 wherein said step b) includes the step of determining a location of an instrument and determining the area of interest based upon the location of the instrument.

15. The method of claim 14 wherein said step b) further includes the step of placing the instrument at the location and manually designating the location as the area of interest.

16. A surgical system comprising:
a CT scanner for taking x-ray update images of a selected area of interest of the patient;
an instrument;
a tracking system for tracking a position and an orientation of the instrument;
a computer storing first data at least partially representing a first area of a patient, the computer generating a 3D model of the area of interest based upon the update images of the area of interest and based upon the first data; and
a display indicating a current position of the instrument relative to the 3D model.

17. They system of claim 16 wherein the tracking system determines a position of the CT scanner at which the CT scanner takes the update images, the computer updating the 3D model based upon the position of the CT scanner.

18. The system of claim 16 wherein the computer determines the area of interest.

19. The system of claim 18 wherein the computer determines the area of interest based upon at least one position of the instrument.

20. The system of claim 19 wherein the instrument is a surgical instrument and wherein the computer determines the area of interest to includes a plurality of positions of the surgical instrument, including the at least one position.

21. The system of claim 19 wherein the instrument includes a switch for selectively designating the area of interest based upon the current position of the instrument.

22. The system of claim 16 wherein the area of interest is smaller than the first area.

23. The system of claim 16 wherein the area of interest is completely contained within the first area.

24. A surgical system comprising:
a fluoroscope for taking x-ray images of a patient;
a tracking system for tracking a position and an orientation of the fluoroscope relative to the patient; and
a computer storing a 3D model of a first area of the patient and determining an area of interest relative to the 3D model to be scanned by the fluoroscope.

25. The system of claim 24 wherein the computer displays the 3D model on a display in order to determine an area of interest.

26. The system of claim 25 wherein the area of interest is selected on the display relative to the 3D model.

27. The system of claim 25 wherein the area of interest to be scanned by the fluoroscope is indicated on the 3D model on the display.

28. The system of claim 24 wherein the fluoroscope is a CT scanner running in a fluoroscopy mode.
